(19) United States
(12) Patent Application Publication
(10) Pub. No.: US 2007/0270838 A1
(43) Pub. Date: Nov. 22, 2007

(54) Dynamic spinal stabilization device with dampener

(75) Inventors: Aurelien Bruneau, Memphis, TN (US); Thomas Carls, Memphis, TN (US); Eric C. Lange, Collierville, TN (US); John D. Pond JR., Germantown, TN (US); Kent Anderson, Memphis, TN (US); Henry Bonin, Memphis, TN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/429,866

(22) Filed: May 8, 2006

(51) Int. Cl. A61F 2/30

(52) U.S. Cl. 606/61

(57) ABSTRACT

Devices and methods for spinal stabilization include first and second anchor assemblies engageable to respective ones of first and second vertebrae and a connecting element engageable to the first and second anchor assemblies. The connecting element includes opposite first and second members and a dampener between the end members that provides a flexible bumper between the end members to provide dynamic stabilization of the spinal column when engaged to the anchor assemblies.

### BACKGROUND

Elongated connecting elements, such as rods, plates, tethers, wires, cables, and other devices have been implanted along the spinal column and connected between two or more anchors engaged between one or more spinal motion segments. Such connecting elements can provide a rigid construct that resists movement of the spinal motion segment in response to spinal loading or movement of the spinal motion segment by the patient. Still other connecting elements are flexible to permit at least limited spinal motion while providing resistance to loading and motion of the spinal motion segment that is the same in both compression and tension. Such flexible connecting elements can be considered to provide dynamic spinal stabilization since at least limited movement of the spinal motion segment is preserved after implantation of the connecting element.

While prior connecting elements provide various spinal stabilization options, there remains a need for connecting elements that can provide dynamic resistance to forces and motion in different directions along the spinal motion segment for dynamic stabilization while maintaining the structural integrity of the connecting element.

### SUMMARY

The present invention generally relates to devices and methods for dynamically stabilizing a spinal column motion segment including at least two vertebrae by engaging a connecting element between the at least two vertebrae. The connecting element includes a flexible and resilient dampener between opposite end members.

According to one aspect, a spinal stabilization system includes first and second anchor assemblies engageable to respective ones of first and second vertebral bodies and an elongated connecting element including opposite first and second end members and a length along a longitudinal axis between the first and second end members sized for positioning between and engaging each of the first and second anchor assemblies when the first and second anchor assemblies are engaged to the respective vertebral bodies. The end members each include a mounting portion and an axially extending rod portion. The connecting element further includes a dampener providing a body extending between and flexibly coupling the first and second end members to one another. The mounting portions of the end members each include a cavity about the longitudinal axis and the flexible body includes end portions extending into each of the cavities and an intermediate portion between the end portions.

According to another aspect, a spinal stabilization system includes first and second anchor assemblies engageable to respective ones of first and second vertebral bodies and an elongated connecting element including a length along a longitudinal axis for engaging the anchor assemblies to stabilize a spinal motion segment. The connecting element includes first and second end members along the longitudinal axis, and each end member includes a rod portion and a mounting portion. The mounting portions each include an elongated shaft extending toward and overlapping the other shaft when assembled, and at least one support member at an end of each of the shafts extending radially outwardly therefrom. The support members define a space therebetween, and the connecting element includes a dampener between the mounting portions flexibly linking the first and second end members to one another. The dampener extends into the space between the support members. When the connecting element is axially tensioned the dampener is compressed between the support members.

According to another aspect, there is provided a connecting element for dynamic spinal stabilization system that includes an elongated body extending along a longitudinal axis. The body includes opposite first and second end members and a dampener extending between and flexibly connecting the end members. The end members each include a rod portion and a mounting portion with the mounting portions each defining a cavity facing one another and extending about the longitudinal axis. The dampener includes end portions extending into each of the cavities and an intermediate portion between the end portions.

According to yet another aspect, a connecting element for dynamic spinal stabilization system includes an elongated body extending along a longitudinal axis. The body includes opposite first and second end members and a dampener extending between and flexibly connecting the end members. The end members each include a rod portion and a mounting portion engaged with an adjacent end of the dampener. The connecting element also includes a linking element engaged to each of the rod portions and extending therebetween along an outer surface of the dampener.

In a further aspect, a method for assembling a connecting element for stabilizing a spinal column segment comprising: providing a first end member with a first rod portion and a first mounting portion at an end of the first rod portion; providing a second end member with a second rod portion and a second mounting portion at an end of the second rod portion; aligning the first and second end members along a longitudinal axis; and molding a dampener between the first and second mounting portions, the dampener having opposite end portions received within cavities defined by each of the mounting portions about the longitudinal axis.

These and other aspects will be discussed further below.

### BRIEF DESCRIPTION OF THE DRAWINGS

FIG. 1 is a posterior elevation view of a spinal column segment and spinal implant system.

FIG. 2 is a longitudinal sectional view of one embodiment connecting element of the spinal implant system of FIG. 1.

FIG. 3 is an elevation view of another embodiment connecting element useable in the spinal implant system of FIG. 1.

FIG. 4 is an elevation view of another embodiment connecting element useable in the spinal implant system of FIG. 1.

FIG. 5 is an elevation view of another embodiment connecting element useable in the spinal implant system of FIG. 1.

FIG. 6 is an elevation view of another embodiment connecting element useable in the spinal implant system of FIG. 1.

FIG. 7 is a longitudinal sectional view of another embodiment connecting element useable in the spinal implant system of FIG. 1.

FIG. 8 is a section view along line 8-8 of FIG. 7 with the dampener removed for clarity in showing the overlapping relationship between the end members.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Devices and methods for providing dynamic stabilization of one or more spinal motion segments are provided. The devices and methods include a connecting element between two or more bone anchor assemblies that can be engaged to at least two or more vertebral bodies of a spinal motion segment. The connecting element extends along a longitudinal axis and includes end members with rod portions at each end engageable to respective ones of the anchor assemblies and a dampener between the end members that allows movement of the vertebrae to which the connecting element is attached. The end members can be configured to interfit with the dampener to provide an integral construct. In further embodiments, a linking element can be provided that extends between the rod portions and externally along the dampener to provide additional structural enhancement of the connecting element. The connecting element can be linear along the longitudinal axis, curved along the longitudinal axis, or include some other non-linear form.

The anchor assemblies discussed herein can be multi-axial or uni-axial in form, and can include an anchor member engageable to a vertebral body and a receiver, post or other device for receiving or engaging a respective end member of the connecting element. The multi-axial anchor assemblies allow the anchor member to be positioned at various angles relative to the connecting element engaging portion of the anchor assembly. The uni-axial anchor assemblies can also provide a fixed positioning of the connecting element engaging portion to the anchor member. The anchor member of the anchor assemblies can form a distal lower portion that is engageable to a vertebral body with the proximal connecting element engaging portion positioned adjacent the vertebral body. In one embodiment, the anchor member is in the form of a bone screw with a threaded shaft and a proximal head that is pivotally captured in the receiver. In other embodiments, the distal anchor member can be in the form of a hook, staple, cable, tether, suture anchor, interbody fusion implant, artificial disc implant, bolt, or other structure engageable to bony tissue. The implant engaging portion can include a receiver with a U-shape, O-shape, or other shape that defines a passage that receives the respective end member of the connecting element therein, thereon, therethrough, or thereover, for example. The connecting element can extend from one or both of the anchor assemblies for securement to one or more additional vertebral bodies.

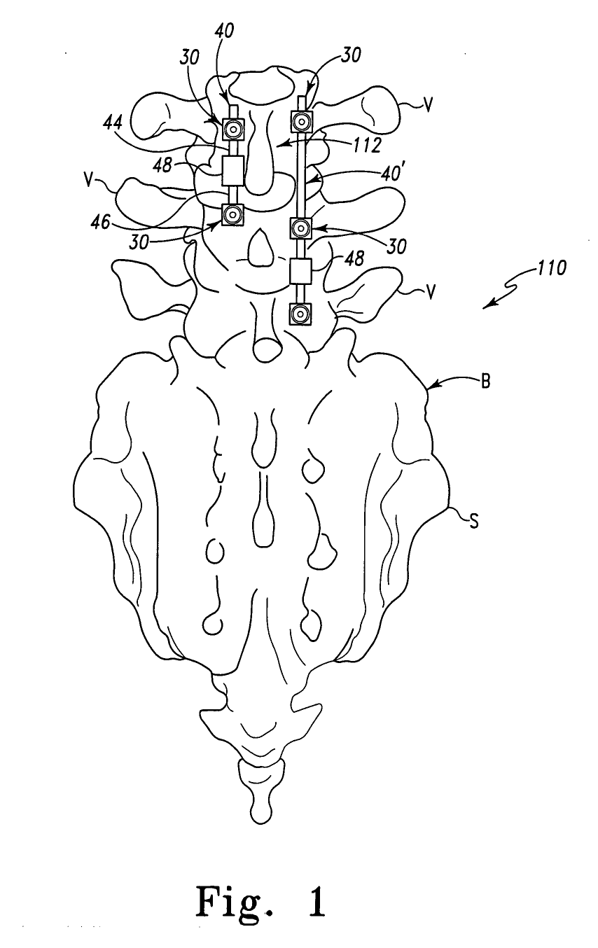

FIG. 1 illustrates a posterior spinal implant system 110 located along a spinal column of a patient. More specifically, implant system 110 can be affixed to bones B of the spinal column segment 112 from a posterior approach, although application in posterior-lateral, lateral, antero-lateral and anterior approaches are also contemplated. Bones B can include the sacrum S and several vertebral bodies V. Implant system 110 generally includes several bone anchor assemblies 30 and elongated connecting elements 40 and 40′ structured to selectively interconnect with bone anchor assemblies 30. Connecting elements 40 may have a dampener 48 between end members 44, 46 and an overall length sized to extend between bone anchor assemblies 30 engaged to least two vertebral bodies V. Connecting element 40′ has a length sized to extend along three or more vertebrae with at least one dampener 48 between adjacent vertebrae. The portions of connecting element 40′ extending between the other vertebrae may include a dampener or may include a rod portion that provides rigid or dynamic stabilization with or without a dampener.

In implant system 110, bone anchor assemblies 30 are affixed to various locations of the spinal column segment 112, such as the pedicles, and interconnected with one or more connecting elements 40, 40′. Other procedures contemplate implant system 110 may be employed at other locations about the spinal column, including anterior, antero-lateral, and lateral locations. Implant system 110 may also be employed in procedures where such locations are combined; e.g. to provide posterior and anterior stabilizations. Implant system 110 may be used for, but is not limited to, treatment of degenerative spondylolisthesis, herniation, degeneration, arthritis, fracture, dislocation, scoliosis, kyphosis, spinal tumor, and/or a failed previous fusion.

FIG. 2 shows an elevation view of one embodiment of connecting element 40, it being understood that connecting element 40′ could be similarly configured albeit with a length to extend between three or more vertebrae as discussed above. Connecting element 40 includes a body 42 extending along a longitudinal axis L between a first end member 44 and an opposite second end member 46. A dampener 48 extends between and connects end members 44, 46. End members 44, 46 can be configured to be engaged to a respective one of the bone anchor assemblies 30 and further configured to be engaged with dampener 48 therebetween. In one embodiment, end members 44, 46 have rod portions 45, 47, respectively, along longitudinal axis L that are in the form of and sized and shaped with a cross-section suitable for a spinal rod system for positioning and implantation along the spinal column of a human patient. In another embodiment, rod portions 45, 47 are each sized with a length along longitudinal axis L that extends from dampener 48 and engages an anchor assembly engaged to an adjacent vertebra.

In another embodiment, one or both of the rod portions 45, 47 has a length along longitudinal axis L that extends between two or more anchor assemblies engaged to two or more adjacent vertebrae, such as shown with connecting element 40′. In such multi-level embodiments, the respective end member 44, 46 can include a cross-section that is constant between adjacent anchor assemblies, or that includes another dampener 48 between anchor assemblies.

Each of the end members 44, 46 further includes a mounting portion 50, 52, respectively, at an end thereof opposite the respective rod portion 45, 47. Mounting portions 50, 52 can each include a cup or bowl type shape opening toward one another along longitudinal axis L with a flange 54, 56 extending about a cavity 58, 60, respectively. Each of the flanges 54, 56 includes a number of holes 62, 64 extending therethrough that are spaced about the perimeter of the respective flanges 54, 56 in a transverse orientation to longitudinal axis L.

Dampener 48 can be provided in the form of a flexible member that provides a shock absorbing effect in transmitting spinal column loads between the anchor assemblies 30 to which it is engaged. Dampener 48 can also permit relative movement between end members 44, 46 to allow motion of the spinal column segment to which connecting element 40 is engaged. End members 44, 46 can be substantially rigid to facilitate percutaneous insertion of connecting element 40 and/or engagement of the end members 44, 46 with anchor assemblies 30. Connecting element 40 can also be inserted and engaged to anchor assemblies 30 in open procedures where the skin and tissue between the anchor assemblies is cut and retracted to allow connecting element placement between the anchor assemblies through the retracted opening.

Various embodiments of connecting element 40 contemplate various techniques for securing end members 44, 46 to dampener 48. In FIG. 2, dampener 48 includes a visco-elastic form that is injection molded between and within mounting portions 50, 52. One suitable material contemplated includes polyurethane rubber, although any suitable, biocompatible material that can be molded with mounting portions 50, 52 is contemplated. Dampener 48 can include opposite end portions 70, 72 with nubs 74 that extend into holes 62, 64 to provide an interface with mounting portions 50, 52 that can resist axial tension forces. Dampener 48 can further include intermediate portion 78 that extends radially outwardly to provide ledges 76, 77 against which the respective adjacent ends of flanges 54, 56 are abuttingly engaged.

When connecting element 40 is subject to tension forces along longitudinal axis L, end members 44, 46 may tend to separate from dampener 48. Nubs 74 can resist this separation by providing a component that extends transversely to the separations force and between the end members 44, 46 and dampener 48. Other embodiments contemplate a linking element that extends between and links first and second end members 44, 46 with one another in a manner that provides resistance to tensile forces that may be exerted on end members 44, 46 in addition to or in lieu of nubs 74.

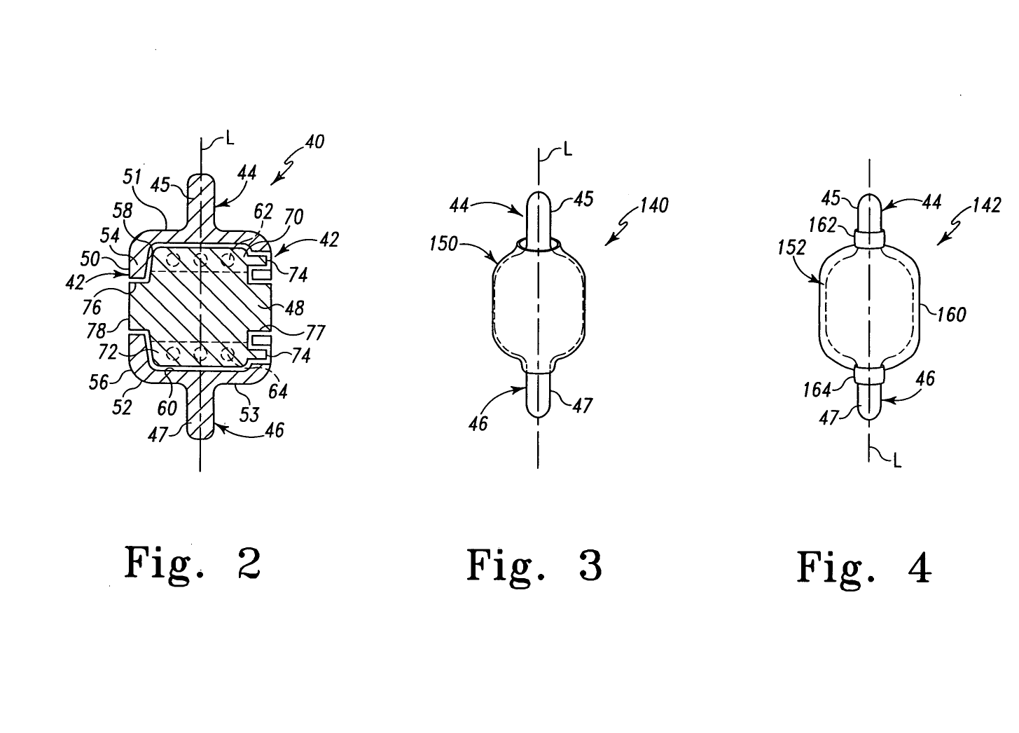

FIG. 3 provides one example of a connecting element 140 that can be similar to connecting element 40, with like elements designated with the same reference numerals. Connecting element 140 includes end members 44, 46 that can be connected to one another with a dampener, like dampener 48 discussed above. There is further provided a linking element 150 that extends between end members 44, 46 and envelopes the dampener 48 and the mounting portions 50, 52 of end members 44, 46. Linking element 150 can be in the form of tubing that is positioned externally about connecting element 140 with rod portions 45, 47 protruding axially therefrom in opposite end openings of the tubing that lie along longitudinal axis L. In the illustrated embodiment, linking element 150 can be heat shrunk to tightly fit about connecting element 140. The tight fit of the tubing about end members 44, 46 and dampener 48 can provide an intimate fit and engagement extend along the oppositely directed faces 51, 53 (FIG. 2) of the mounting portions 50, 52 that are transversely oriented to longitudinal axis L, resisting movement of the end members 44, 46 away from one another and away from the dampener.

FIG. 4 provides an example of another embodiment linking element 152 for connecting element 142. Connecting element 142 is similar to connecting element 40 and like elements are designated with the same reference numerals. Linking element 152 includes a bag-like body 160 extending between opposite ends 162, 164. Ends 162, 164 can be crimped or otherwise secured about the respective rod portions 45, 47 of end members 44, 46. The bag-like body 160 need not tightly fit around end members 44, 46 and dampener 48, although such is not precluded. A loose fit allows dampener 48 to flex in compression and can provide for some lengthening of the space between end members 44, 46 until body 160 is tightly stretched. The engagement of the ends 162, 164 about rod portions 45, 47 maintains linking element 152 in engagement with connecting element 142 to resist separation of the end members from the dampener as connecting element 142 is subjected to axial tension, compression and torsional forces.

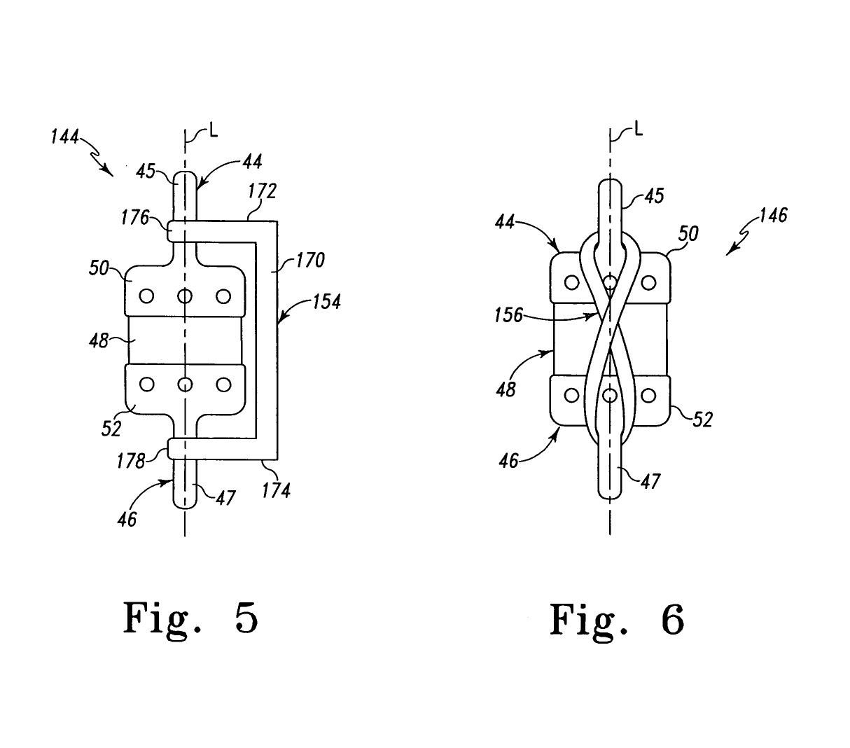

Referring now to FIG. 5, there is shown another embodiment connecting element 144 that includes an external linking element 154 extending between and linking rod portions 45, 47 of end members 44, 46. Linking element 154 includes a longitudinal link or bar 170 extending along longitudinal axis L between connector portions 172, 174. Connector portions 172, 174 extend from respective ends of bar 172 to an engaging end 176, 178 engaged to respective ones of the rod portions 45, 47. Engaging ends 176, 178 can include a passage through which rod portions 45, 47 extend and can axially move. Axial movement is restrained when engaging ends 176, 178 are contacted by the mounting portions 50, 52 when connecting element 144 is subjected to tension loading. Bar 170 can be rigid to prevent any movement of end members 44, 46 away from one another when engaging ends 176, 178 are contacted by mounting portions 50, 52. A rigid bar 170 can also contact the anchor assemblies to provide a limit to the movement of the anchor assemblies 30 toward one another under compression. Bar 170 can alternatively be flexible and elastic to flex in response to compression and tension loading. A flexible bar 170 can be sufficiently resistant to movement and loading in tension to prevent end members 44, 46 from separating from dampener 48.

Referring now to FIG. 6, there is shown another embodiment connecting element 146 that includes an external linking element 156. Linking element 156 can be in the form of a band that is looped around the respective rod portions 45, 47 of end members 44, 46 to provide axial restraint in tension of the end members 44, 46 relative to the dampener 48. Linking element 156 can provide a cross-over at its mid-portion to facilitate maintaining the band in position along the connecting element 146. The ends of the linking element can be crimped or spliced to form a loop, or the band can be a continuous type loop without overlapping ends. It is also contemplated that linking element 156 can be twisted to provide multiple crossovers to shorten its length or to provide additional tension restraint. It is also contemplated that multiple linking elements 156 can be provided as may be desired for additional restraint of axial forces.

The linking elements can provide the connecting element with a stiffness that provides more resistance to spinal motion that creates axial tension loading without resisting or hindering spinal motion that results in axial compression loading, although it is contemplated that some compression loading resistance is not precluded. Accordingly, spinal motion can be preserved while more effectively limiting tension or movement of the adjacent vertebral bodies away from one another while maintaining the connecting element as a functioning unit and resisting separate of one or both of the end members from the dampener.

The end members and/or linking elements can be made from nitinol, titanium, stainless steel, or other biocompatible metals and alloys thereof. The end members and/or linking elements can also be made from PEEK or other polymer material that is biocompatible. The linking element can be made from a material that is the same as or that differs from the material of the end members. The linking element can be a rod, cord, rope, wire, tether, belt, band, ribbon, braid, suture, bar, bag, sack, shrink wrap, sleeve, tube, or include any other suitable form.

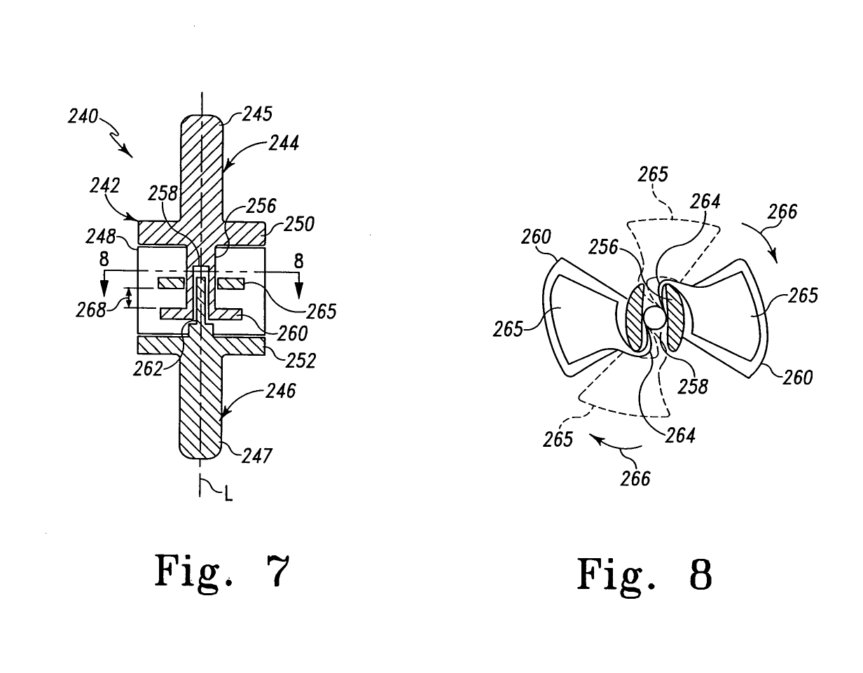

Other embodiments contemplate that dampener 48 is molded about components of the end portions that extend within the dampener and provide platforms or surfaces that compress at least a portion of dampener 48 when the connecting element is subjected to either of axial tension or axial compression loading. For example, FIG. 7 shows a connecting element 240 having a body 242 with a first end member 244 and a second end member 246. End members 244, 246 each include a rod portion 245, 247 extending in opposite directions from one another along longitudinal axis L for engagement with anchor assemblies as discussed above with respect to rod portions 45, 47. Connecting element 240 further includes a dampener 248 providing a flexible bumper or stabilizer between end members 244, 246 that allows movement of the spinal motion segment or segments to which connecting element 240 is attached while maintaining separation of the adjacent vertebrae.

End members 244, 246 each further include a mounting portion 250, 252 to which dampener 248 is mounted between end members 244, 246. Mounting portion 250 includes an outer shaft 256 extending axially and oppositely of rod portion 245. Outer shaft 256 includes an axial bore 258 and opposite support members 260 extending radially outwardly from outer shaft 256. Mounting portion 252 includes an inner shaft 262 extending axially and in an opposite direction from rod portion 246 and into axial bore 258 of outer shaft 256. Axial bore 258 is slotted so that wings 264 extending from inner shaft 262 can extend through the slots. Support members 265 extend radially outwardly from the respective wings 264.

In order to assemble end members 244, 246, inner shaft 262 is inserted into outer shaft 256 with support members 265 in a first position, as indicated by the dashed lines in FIG. 8. Mounting portion 252 can then be rotated relative to mounting portion 250 as indicated by arrows 266 to a position where support members 265 are aligned radially with support members 260, as shown in solid lines in FIG. 8. Outer shaft 256 can be keyed to receive wings 264 in the rotated position to prevent axial displacement of the mounting portions 250, 252 relative to one another. Dampener 248 can then be formed or molded about and between mounting portions 250, 252 to provide the flexible bumper therebetween.

In the aligned orientation of support members 260, 265, an axial space 268 (FIG. 7) is formed between the support members 260, 265 that receives a portion of dampener 248 therein. Accordingly, at least this portion of dampener 248 is compressed between support members 260, 265 even when connecting element 240 is placed in axial tension loading conditions. Dampener 248 thus provides resistance to displacement of end members 244, 246 away from one another under axial tension and maintain connecting element 240 in an intact condition.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal stabilization system, comprising:
first and second anchor assemblies engageable to respective ones of first and second vertebral bodies; and
an elongated connecting element including opposite first and second end members and a length along a longitudinal axis between said first and second end members sized for positioning between and engaging each of said first and second anchor assemblies when said first and second anchor assemblies are engaged to the respective vertebral bodies, wherein each of said end members includes a mounting portion and an axially extending rod portion, said connecting element further including a dampener with a body extending between and flexibly coupling said first and second end members to one another, wherein said mounting portions of said end members each include a cavity about said longitudinal axis and said flexible body includes end portions extending into each of said cavities and an intermediate portion between said end portions.

2. The system of claim 1, wherein said rod portions of said end members each extend away from one another along said longitudinal axis and at least one of said rod portions has a length sized to extend between at least two vertebral bodies.

3. The system of claim 1, wherein each of said cavities is defined by a flange of said respective mounting portion that extends about said cavity, said flanges each including a number of holes extending therethrough transversely to said longitudinal axis.

4. The system of claim 3, wherein said end portions of said body of said dampener each include nubs extending into said holes to axially restrain said end members to said dampener.

5. The system of claim 3, wherein said flanges each include an endwall extending around said cavity about said longitudinal axis and said intermediate portion includes a ledge extending thereabout adjacent each of said end portions, said endwalls of said flanges being positioned in abutting engagement with an adjacent one of said ledges.

6. The system of claim 1, further comprising a linking element engaged to each of said end members and extending along an outer side of said dampener between said end members.

7. The system of claim 6, wherein said linking element includes an elongated tube in form fitting engagement about said mounting portions and said dampener, said tube including opposite ends that engage an adjacent face of a respective one of said mounting portions, said faces of said mounting portions being oriented transversely to said longitudinal axis and facing away from one another.

8. The system of claim 6, wherein said linking element includes a bag with a body extending between opposite ends, said body being positioned about said mounting portions and said dampener with said opposite ends engaged to respective ones of said rod portions of said end members.

9. The system of claim 6, wherein said linking element includes a bar extending along said longitudinal axis between opposite connector portions, said connector portions extending between and engaging a respective one of said rod portions to said bar.

10. The system of claim 9, wherein said rod portions are axially movable in said connector portions relative to said bar in response to compression of said dampener.

11. The system of claim 10, wherein said mounting portions contact said connector portions so that said bar limits displacement of said end members away from one another when said connecting element is under axial tension loading.

12. The system of claim 6, wherein said linking element includes a band extending about and linking each of said rod portions to one another.

13. The system of claim 12, wherein said band forms a loop between said end portions having at least one cross-over along said dampener.

14. A spinal stabilization system, comprising:
first and second anchor assemblies engageable to respective ones of first and second vertebral bodies;
an elongated connecting element including a length along a longitudinal axis sized for positioning between and engaging each of said first and second anchor assemblies when said first and second anchor assemblies are engaged to the respective vertebral bodies, wherein said connecting element includes:
first and second end members along said longitudinal axis, said end members each including a rod portion and a mounting portion, said mounting portions each including an elongated shaft extending toward and overlapping the other shaft along said longitudinal axis and further including at least one support member adjacent an end of each of said shafts extending outwardly therefrom, said support members defining a space therebetween; and
a dampener between said mounting portions flexibly linking said first and second end members to one another, said dampener extending into said space between said support members, wherein when said connecting element is axially tensioned said dampener is compressed between said support members.

15. The system of claim 14, wherein said shaft of said mounting portion of said first end member is axially received in a bore in said shaft of said mounting portion of said second end member.

16. The system of claim 15, wherein said at least one support member of said mounting portion of said first end member is connected to said shaft thereof with a wing extending between said shaft and said at least one support member, said wing extending through a slot in said shaft of said mounting portion of said second end member.

17. The system of claim 14, wherein said dampener is comprised of a flexible, resilient material.

18. A connecting element for dynamic spinal stabilization system, comprising:
an elongated body extending along a longitudinal axis and including opposite first and second end members and a dampener extending between and flexibly connecting said end members, wherein said end members each include a rod portion and a mounting portion with said mounting portions each defining a cavity facing one another and extending about said longitudinal axis, wherein said dampener includes end portions extending into each of said cavities and an intermediate portion between said end portions.

19. The connecting element of claim 18, wherein each of said cavities is defined by a flange of said respective mounting portion extending about said cavity, said flanges each including a number of holes extending therethrough transversely to said longitudinal axis.

20. The connecting element of claim 19, wherein said end portions of said body of said dampener each include nubs extending into said holes to axially restrain said end members to said dampener.

21. The connecting element of claim 19, wherein said flanges each include an endwall extending about said longitudinal axis and said intermediate portion includes a ledge extending thereabout adjacent each of said end portions, said endwalls of said flanges each being positioned in abutting engagement with an adjacent one of said ledges.

22. A connecting element for dynamic spinal stabilization system, comprising:
an elongated body extending along a longitudinal axis and including opposite first and second end members and a dampener extending between and flexibly connecting said end members, wherein said end members each include a rod portion and a mounting portion engaged with an adjacent end of said dampener, and further comprising a linking element engaged to each of said rod portions and extending therebetween along an outer surface of said dampener.

23. The connecting element of claim 22, wherein said linking element includes an elongated tube in form fitting engagement about said mounting portions and said dampener, said tube including opposite ends that engage an adjacent face of a respective one of said mounting portions, said faces of said mounting portions being oriented transversely to said longitudinal axis and facing away from one another.

24. The connecting element of claim 22, wherein said linking element includes a bag with a body extending between opposite ends, said body being positioned about said mounting portions and said dampener with said opposite ends engaged to respective ones of said rod portions of said end members.

25. The connecting element of claim 22, wherein said linking element includes a bar extending along said longitudinal axis between opposite connector portions, said connector portions extending between and engaging a respective one of said rod portions to said bar.

26. The connecting element of claim 25, wherein said rod portions are axially movable in said connector portions relative to said bar in response to compression of said dampener.

27. The connecting element of claim 26, wherein said mounting portions contact said connector portions so that said bar limits displacement of said end members away from one another when said connecting element is under axial tension loading.

28. The connecting element of claim 22, wherein said linking element includes a band extending about and linking each of said rod portions to one another.

29. The connecting element of claim 28, wherein said band forms a loop between said rod portions having at least one cross-over along said dampener.

30. A method for assembling a connecting element for stabilizing a spinal column segment, comprising:
providing a first end member with a first rod portion and a first mounting portion at an end of the first rod portion;
providing a second end member with a second rod portion and a second mounting portion at an end of the second rod portion;
aligning the first and second end members along a longitudinal axis; and
molding a dampener between the first and second mounting portions, the dampener having opposite end portions received within cavities defined by each of the mounting portions about the longitudinal axis.

31. The method of claim 30, wherein the first and second mounting portions each include a flange extending about the cavity, each of the flanges including a plurality of holes extending transversely to the longitudinal axis, and molding the dampener includes molding nubs into the holes to axially restrain the end members to the dampener.

32. The method of claim 31, further comprising engaging a linking element between each of the first and second end members with the linking element extending externally along the dampener.

33. The method of claim 32, wherein engaging the linking element includes engaging opposite ends of the linking element to each of the first and second rod portions and surrounding the dampener and the first and second mounting portions with a bag extending between the opposite ends.

34. The method of claim 32, wherein engaging the linking element includes tightly fitting a tubular sleeve about the dampener with opposite ends of the sleeve extending along faces of the first and second mounting portions, wherein the faces of the mounting portions extend transversely to the longitudinal axis and face in opposite directions from one another.

35. The method of claim 32, wherein engaging the linking element includes looping opposite ends of a band about respective ones of the first and second rod portions.

36. The method of claim 32, wherein engaging the linking element includes positioning the first and second rod portions through connector portions of the linking element, the linking element further including a bar extending longitudinally between the connector portions and the connector portions extend transversely from opposite ends of the bar to the respective rod portions.

37. The method of claim 30, wherein aligning the first and second end members includes positioning first and second shafts extending along respective ones of the first and second end members in overlapping relation to one another along the longitudinal axis.

38. The method of claim 37, wherein each of the first and second shafts includes at least one support member extending outwardly therefrom in a transverse orientation to the longitudinal axis, wherein the support members define a space therebetween when the first and second end members are aligned, and molding the dampener includes molding the dampener in the space.
